(19) United States
(12) Patent Application Publication
(10) Pub. No.: US 2025/0064474 A1
(43) Pub. Date: Feb. 27, 2025

(54) PERICARDICAL TRANSECTION DEVICES AND METHOD

(71) Applicant: EDWARDS LIFESCIENCES CORPORATION, Irvine, CA (US)

(72) Inventors: Yuval Kasher, Kfar Shmuel (IL); Dikla Kersh, Karkur (IL)

(21) Appl. No.: 18/940,504

(22) Filed: Nov. 7, 2024

Related U.S. Application Data
(63) Continuation of application No. PCT/US2023/022928, filed on May 19, 2023.
(60) Provisional application No. 63/346,314, filed on May 26, 2022.

(51) Int. Cl. A61B 17/32; A61B 17/00; A61B 17/3209; A61B 17/3211; A61B 18/00; A61B 18/14

(52) U.S. Cl. CPC A61B 17/320016; A61B 17/3209; A61B 18/1492; A61B 2017/00247; A61B 2017/32113; A61B 2018/00351; A61B 2018/00601

(57) ABSTRACT

Exemplary transection devices for making incisions through the pericardial membrane or parietal layer of the pericardium are described. These examples share the characteristic that they are deployed intravascularly through the RAA, IVC, SVC, or via a subxiphoid approach. One example is a pericardial tissue transection device comprising an elongated body with a proximal end and a distal end and extending along a longitudinal length. An incision assembly is coupled to the distal end of the elongated body. The incision assembly comprises an incision member having first and second ends each coupled to the incision assembly and aligned with the longitudinal length of the elongate body. At least a portion of the incision member is structured to reversibly extend laterally from the elongated body.

### RELATED APPLICATIONS

This application is a continuation application of International Patent Application No. PCT/US2023/022928, filed May 19, 2023, which claims the benefit of U.S. Provisional Application No. 63/346,314, filed on May 26, 2022. Each of the foregoing patent applications are hereby incorporated by reference in their entireties.

### TECHNICAL FIELD

This disclosure is directed to methods for treating heart failure, for example, heart failure with preserved ejection fraction (HFpEF) or reduced injection fraction (HFrEF) by providing pericardial transection devices that introduce one or more incision lengths in a pericardium, e.g., a pericardial layer, fibrous layer, and/or adipose tissue.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In a first example, a pericardial tissue transection device is provided, the device comprising an elongated body with a proximal end and a distal end, and a longitudinal axis, and an incision assembly coupled to the distal end, the incision assembly comprising an incision member aligned with the longitudinal axis, at least a portion of the incision member structured to reversibly extend laterally from the incision assembly to engage and to cut pericardial tissue.

In one aspect, alone or in combination with the prior aspect, the pericardial tissue transection device further comprises an actuator operably coupled to the incision assembly, the actuator traversing parallel with the longitudinal axis from a first configuration disengaged with the incision member to a second configuration engaged with the incision member such that in the second configuration the incision member is laterally extended.

In one aspect, alone or in combination with any of the previous aspects, the actuator is a rigid rod or wire.

In one aspect, alone or in combination with any of the previous aspects, the incision member is generally planar.

In one aspect, alone or in combination with any of the previous aspects, the incision member comprises a first end and a second end, the first and the second ends coupled to the incision assembly.

In one aspect, alone or in combination with any of the previous aspects, at least a portion of the incision member is a sharpened edge.

In one aspect, alone or in combination with any of the previous aspects, at least a portion of the incision member is energizable with electrical current or radio frequency energy sufficient to separate pericardial tissue.

In one aspect, alone or in combination with any of the previous aspects, the incision member is a sharpened edge with at least a portion thereof energizable with electrical current or radio frequency energy sufficient to separate pericardial tissue.

In one aspect, alone or in combination with any of the previous aspects, the incision member is rigidly flexible.

In one aspect, alone or in combination with any of the previous aspects, the pericardial tissue transection further comprises stabilizing members structured to reversibly extend laterally from the elongated body.

In one aspect, alone or in combination with any of the previous aspects, the pericardial tissue transection device further comprises a retractable sheath covering the incision assembly.

In one aspect, alone or in combination with any of the previous aspects, at least a portion of the retractable sheath comprises radiopaque material.

In one aspect, alone or in combination with any of the previous aspects, the pericardial tissue transection device further comprises an introducer/dilator adjacent the distal end of the incision assembly.

In one aspect, alone or in combination with any of the previous aspects, the introducer/dilator receives a guidewire.

In one aspect, alone or in combination with any of the previous aspects, at least a portion of the introducer/dilator comprises radiopaque material.

In one aspect, alone or in combination with any of the previous aspects, the pericardial tissue transection device further comprises visualization means.

In one aspect, alone or in combination with any of the previous aspects, the pericardial transection device is sterilized.

In a second example, a method of incising pericardial tissue in a subject in need thereof is provided, the method comprising providing a pericardial device of any one of the previous examples, introducing the pericardial device to a pericardial cavity, and incising at least a portion of a parietal layer of a pericardium along a length and a path

In one aspect, alone or in combination with any of the previous aspects, the pericardial device is introduced subxiphoidally.

In one aspect, alone or in combination with any of the previous aspects, the pericardial device is introduced transvascularly.

In one aspect, alone or in combination with any of the previous aspects, the pericardial device is introduced transvascularly via the Superior Vena Cava.

In one aspect, alone or in combination with any of the previous aspects, the pericardial device is introduced transvascularly via the Inferior Vena Cava.

In one aspect, alone or in combination with any of the previous aspects, the incising of at least a portion of the parietal layer is by reverse cutting along a path and a length.

In one aspect, alone or in combination with any of the previous aspects, the method further comprises repeating the step of incising the pericardial cavity along a different length, a different path, or a different length and a different path.

In one aspect, alone or in combination with any of the previous aspects, the method further comprises, after the introducing step and before the incising step, puncturing out of the pericardial cavity and exposing the incision portion.

In another example, a method of manipulating a transection device is provided, the method comprising providing a pericardial device of any of the embodiments disclosed herein engaged with a controller; and controlling at least one of rotation of the elongated body, extension and retraction of the incision device or stabilizing members, and/or a source of energy to the incision device with the controller.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1C of FIG. 1A depicting the serosal, visceral, fibrous layers and adipose tissue of the parietal pericardium, including the pericardial cavity.

FIGS. 2A-2E depict an example of a pericardial layer tissue transection device as disclosed or described herein.

FIGS. 3A-3C depict a visualization system for use in a multi-lumen device in combination with the presently disclosed pericardial layer tissue transection devices.

FIG. 4 depicts an exemplary controller device for delivering the presently disclosed transection devices, as disclosed and described herein.

FIG. 5 is a simplified diagram of a multi-lumen approach to the pericardial cavity, as disclosed and described herein.

FIG. 6 is a simplified diagram of an alternative multi-lumen approach to the pericardial cavity, as disclosed and described herein.

FIG. 7 is a simplified diagram of a parietal layer incision length and cut path as disclosed and described herein.

### DETAILED DESCRIPTION

Several exemplary devices for making incisions through the pericardial membrane or parietal layer of the pericardium are described. These examples share the characteristic that they are deployed intravascularly through the RAA, IVC, SVC, or via a subxiphoid approach.

As used herein, the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein, the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein, the phrase “cutting surface” is inclusive of one or more of an edge of a sharpened blade or the surface of an electrode structured to receive sufficient current or radio frequency energy (RF) to ablate, burn, vaporize, or separate tissue. A cutting surface as used herein is inclusive of both a sharpened edge and an electrode.

As used herein, the phrase “reverse cutting” and “pull-back cutting” are used interchangeably and refer to methods involving the presentation of a cutting surface to tissue, the cutting surface adjacent a distal end of a multi-lumen device or catheter, and the application of a directional force sufficient to cut or separate the tissue, the force being substantially in a direction towards the proximal end of the multi-lumen device or catheter, for example, by pulling the multi-lumen device or catheter while the cutting surface is engaged with the tissue.

As used herein, the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and/or the like.

As used herein, the phrase “incision length” is inclusive of a non-zero distance of a cut or incision, for example, beginning at a first point, e.g., a target point, and terminating at a second point, e.g., an end point. An incision length includes linear, non-linear, and/or a plurality of linear and/or non-linear lengths that intersect or do not intersect about a curved or non-planar surface, such as a heart.

As used herein, the phrase “reducing pressure” and “reducing restraint” are inclusive of their ordinary and customary meaning of one to ordinary skill in medical and surgical arts.

As used herein, the phrase “preserved ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein, the phrase “heart dysfunction” is inclusive of the ordinary and customary meaning to one of ordinary skill in medical and surgical arts, for example, heart failure or congestive heart failure.

As used herein, the phrase “pericardial transection device” is inclusive of a device with an incision surface, for example, an edge of a blade or a surface of an energized electrode.

As used herein, the phrase “pericardial incision assembly” and “incision assembly” are used interchangeable and refer to an assemblage that includes a pericardial transection device.

As used herein, the phrase “multi-lumen device” is inclusive of a catheter structured with at least one lumen comprising a medical instrument, device, or component thereof, for example, a pericardial transection device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein, the phrase “nerve stimulation device” is inclusive of a device capable of applying an electrical potential to a nerve to cause an observable effect that is directly or indirectly correlated to the applied potential, for example, a pacing probe stimulating a phrenic nerve to cause an observable breathing disruption.

As used herein, the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein, the term “actuator” is inclusive of a mechanism for triggering an action.

As used herein, the term “controller” is inclusive of a device having an actuator.

As used herein, the phrase “biasing member” is inclusive of a device configurable in a stored energy state and a released energy state, for example, a spring.

As used herein, the phrase “stabilizing member” is inclusive of a device configurable to impart stability and/or securement of a device to or within a structure, such as for example, stabilizing or securing a cutting surface positioned in a pericardial cavity from rolling, twisting, buckling, and/or oscillating prior to and/or during use.

As used herein, the phrase “puncturing tip” is inclusive of an atraumatic object suitable for puncturing or penetrating tissue without substantial trauma to or bleeding from the vicinity of the picture or penetration.

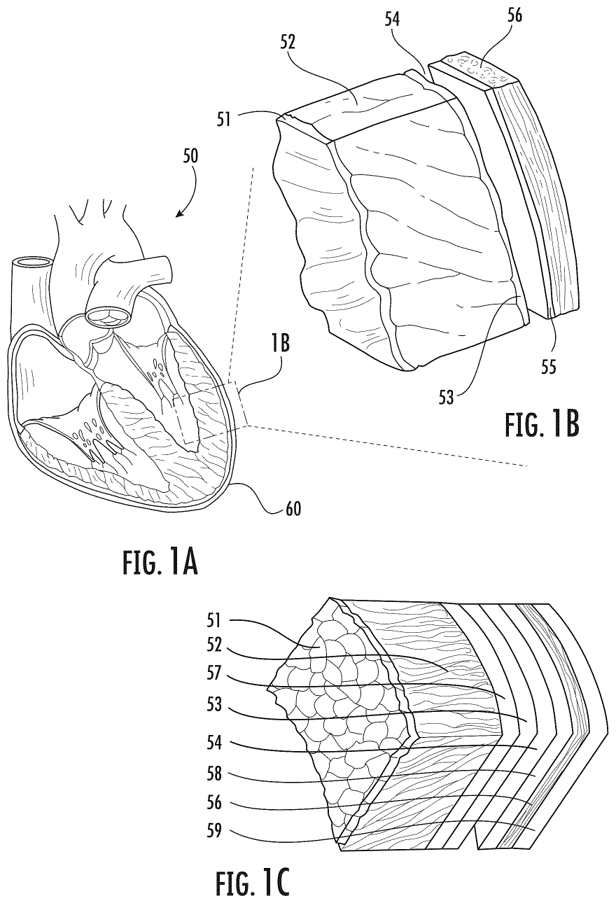

With reference to FIG. 1A and section 1B, layers of a heart wall of a heart 50, from inside-out, being the endocardium 51, the myocardium 52, epicardial adipose tissue 57, the visceral layer 53 of the serous pericardium, the pericardial cavity 54, the parietal layer 58 of the serous pericardium 55, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

The presently disclosed pericardial tissue transection devices includes a perforating or puncturing portion designed to initially puncture the pericardial membrane. A guidewire, knife, needle, microneedle, or electrical current may be used to form the perforation or puncturing of the pericardial membrane to allow access of the pericardial transection device to the pericardial cavity. Once the pericardial membrane is punctured, an incision assembly adjacent a distal end of a catheter or multi-lumen is manipulated to a location within the pericardial cavity, an incision member is allowed to engage with the pericardial tissue, and an incision is created upon retraction of the pericardial tissue transection device towards the point of entry into the pericardial membrane. The incision member may alternatively, or in combination with a sharp edge, utilize RF energy to facilitate ease of incising and for providing some hemostasis of the pericardial membrane.

Several example pericardial tissue transection devices are illustrated in the attached figures. Hereinafter, the phrase “pericardial tissue transection device” and “transection device” shall be used interchangeably. Each transection device would be first introduced into the pericardial space via a transvascular or subxiphoid approach.

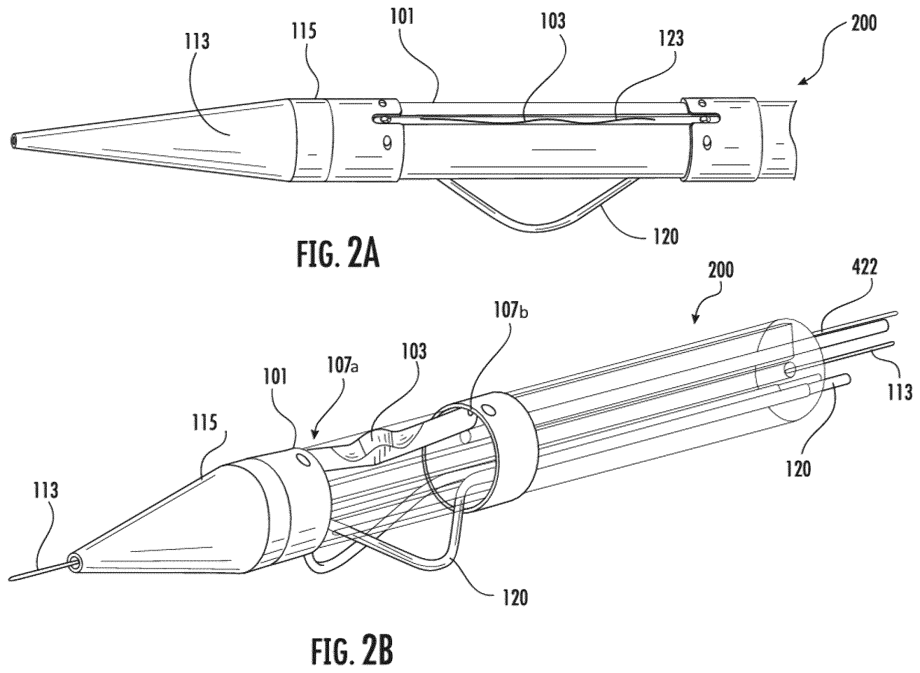

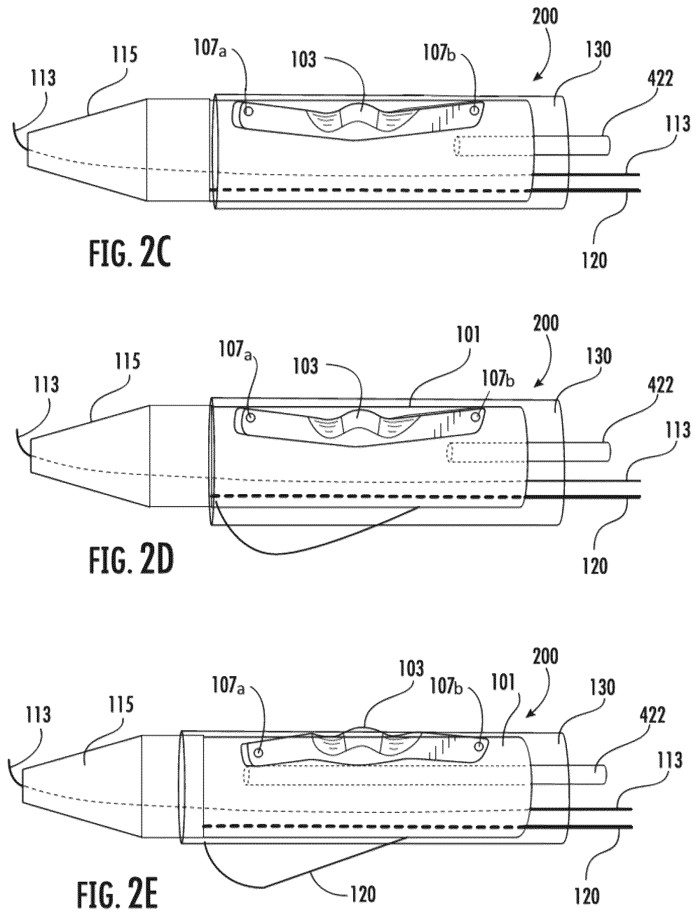

With reference to FIGS. 2A-2E, a first exemplary transection device 200 is shown. Transection device 200 includes a selectively active mechanical sharp edge or RF blade. Transection device 200 is also structured for insertion into the pericardial cavity 54 and manipulated to a location where incising is to begin as described above. Thus, with reference to FIGS. 2A-2E, the transection device 200 comprises an elongated body 129 with proximal end and a distal end, and a longitudinal axis and an incision assembly 101 coupled to the distal end. Transection device 200 is shown with introducer/dilator 115 adjacent the distal end of the incision assembly 101, where the introducer/dilator is structured to receive a guidewire 113. The introducer/dilator 115 may initially follow an initial perforation or puncture the pericardial membrane to allow access of the pericardial transection device 200 to the pericardial cavity. This operation may similarly be performed by or with the assistance of a guidewire 113 or electrical current.

The transection device 200 further includes an incision assembly 101 coupled to the distal end of the elongated body 129. The incision assembly 101 comprises an opening 123 that allows the incision member 103 to, in operation, extend laterally from elongated body 129. The lateral movement of the incision member 103 extends beyond a peripheral edge (e.g., the outer diameter of the elongated body 129) to expose the incision member 103 to an external environment of the transection device 200 and into contact with pericardial tissue. In one example, the incision member 103 is aligned with the longitudinal axis and at least a portion of the incision member 103 structured to reversibly extend laterally from the opening 123 to engage and to cut pericardial tissue.

As shown in FIGS. 2B-2E, the transection device 200 further comprises an actuator 422 operably coupled to the incision assembly 101 and structured to cause the incision member 103 to extend laterally from the elongated body 129 to be exposed (e.g., via the opening 123) to pericardial tissue. The actuator 422 is structured to translate or otherwise traverse parallel with respect to the longitudinal axis of the elongated body 129. As shown in FIGS. 2C and 2D, the actuator 422 may move initially from a first configuration in which the actuator is disengaged from the incision member 103. Due to this disengagement, the incision assembly 101 (e.g., the incision member 103) remains positioned within the elongated body 129. As shown in FIG. 2E, the actuator 422 translates parallel with respect to the longitudinal axis of the elongated body 129 to a second configuration in which the actuator 422 engages the incision assembly 101. This engagement with the incision assembly 101 causes the incision member 103 to laterally extended through the opening 123 to engage and to cut pericardial tissue as described above.

The incision member 103 can be generally planar. The incision member 103 can define a first end and a second end that are coupled to the incision assembly 101. The first and the second ends can be rotationally engaged at points 107a, 107b with the catheter 129 (e.g., at respective collars). In one example, the incision member 103 is rigidly flexible. For example, the incision member 103 can include a central portion that is flexible relative to the first and second ends. When the actuator 422 engages the incision member 103, at least a portion of the incision member 103 deflects or otherwise flexes about the points 107a, 107b to extend laterally outwardly from the elongated body 129 through the opening 123. Upon disengagement with the actuator 422, the incision member 103 returns to an initial or resting configuration (e.g., the first configuration) within the elongated body 129 (e.g., below the opening 123 of the incision assembly 101). Although described herein with reference to deflection of the incision member 103 about the points 107a, 107b, the present disclosure contemplates the incision assembly 101 may include any mechanism by which the incision member 103 extends beyond a peripheral edge of the elongated body 129.

In other examples, the incision member 103 of the transection device 200 may not flex to an engaged position (e.g., the second configuration). Alternatively, the incision member 103 may move only between two defined positions, for example, a deployed position (e.g., the second configuration) and a collapsed position (e.g., the first configuration). In such an example, the incision member 103 may be sufficiently stiff to resist further movement. Such an example incision member 103 may be useful in implementations in which the transection device 200 is used to lacerate tissues that are relatively movable, such as fat tissue about the pericardium, as the application of force to the transection device 200 may be undesirable as it may result in unintended movement of said fat tissue.

In order to cut pericardial tissue as described herein, in some examples, at least a portion of the incision member 103 is a sharpened edge. Additionally or alternatively, at least a portion of the incision member 103 is energizable with electrical current or radio frequency energy sufficient to separate pericardial tissue. In one example, the incision member 103 is a sharpened edge with at least a portion thereof energizable with electrical current or radio frequency energy sufficient to separate pericardial tissue. In any embodiments, the present disclosure contemplates that the incision member 103 comprises any element or mechanism by which tissue is ablated, burned, vaporized, or otherwise separated. In some examples, the incision member 103 comprises a radio frequency (RF), alone or in conjunction with the incision member 103 examples described above. The RF wire, for example, is selectively insulated along various lengths so that the insulated, or non-cutting, areas maintain contact and position within the pericardial space. Furthermore, several RF wires may be used, for example two, three, four, or more, in which a determined number of the wires fix the incision member 103 into position and one (or more) of the wires that contact the pericardial tissue are activated to make the desired cut. This method can be further adapted to provide a specific geometry or pattern of cut as desired to achieve optimal reductions in the intracardiac pressures.

As shown, transection device 200 further comprise stabilizing members 120 structured to reversibly extend laterally from the elongated body 129. The stabilizing members 120 may be structured to translate or otherwise traverse parallel with respect to the longitudinal axis of the elongated body 129 and, in some embodiments, parallel with respect to the actuator 422. As shown in FIG. 2C, the stabilizing members 120 may move similar to the actuator 422 from a first configuration in which the stabilizing members 120 are disposed within the elongated body 129. As shown in FIGS. 2D-2E, the stabilizing members 120, in operation, move to a second configuration in which the stabilizing members 120 extend laterally outward from the elongated body 129, such as via a respective opening in the elongated body 129 aligned with the movement of the stabilizing members 120. The stabilizing members 120 similarly extend beyond a peripheral edge of the elongated body 129 to contact pericardial tissue and prevent or otherwise reduce the ability of the transection device 200 to rotate. The relative position of the stabilizing members 120 (e.g., radially disposed about the elongated body 129 relative the incision member 103) may vary based upon the number of stabilizing members 120, the depth of the intended incision, among other factors. The present disclosure contemplates that the stabilizing members 120 may include any mechanism for extending beyond a peripheral edge of the elongated body 129, such as opposite the incision member 103. The one or two stabilizing members 120 may be independently user controlled by advancing actuating proximal ends of the stabilizing members 120 distally. In one example, two or more stabilizing members 120 are positioned radially about the assembly. In one example, two or more stabilizing members 120 are positioned radially about the assembly about 120 degrees apart. In one example, the two or more stabilizing members 120 are aligned opposite the incision assembly 101. In one example, the two or more stabilizing members 120 are offset longitudinally from the incision assembly 101 to minimize or eliminate pushing the device through the newly cut slit in the parietal layer 55 of the pericardium 60 just as it is formed.

The one or more stabilizing members 120 can comprise flexible rods. The stabilizing members 120 can extend radially outwardly from the catheter. The stabilizing members 120 can each include a distal end fixed with a distal end of the catheter (i.e. distal to the incision assembly 101), an exposed portion aligned with the incision assembly 101 at the respective openings and/or a proximal portion contained within a lumen of the catheter. Advancement of each of the proximal ends can cause the exposed portions to bend radially outwardly. The exposed portions can include one or more planar faces (e.g., strips) such that the bending can be controlled to extend at a fixed angle relative to the catheter. Alternatively, the stabilizing members 120 can be inflatable structures, such as balloons that can be inflated with air or liquid (saline).

The transection device 200 a shown further comprises a retractable sheath 130 covering or surrounding the incision assembly 101. In some examples, the sheath further comprises slits, recesses, grooves, and/or the like each of which are aligned with the laterally protruding elements described herein (e.g., the incision member 103 and/or the exposed portions of the stabilizing members 120). In an example, the sheath 130 comprises slits within which the incision member 103 and/or the stabilizing members 120 translate to prevent unintended circumferential movement of these elements. In any example, the transection device 200 additionally or alternatively includes a plurality of lumens or other equivalent structures structured to receive or otherwise support various elements and members described herein. For example, these one or more lumen structures may cover or support the incision assembly 101 and/or the stabilizing members 120, and/or may operate to provide contrast fluid, electrical current, and/or the like. In some examples, at least a portion of the retractable sheath 130 comprises radiopaque material randomly distributed or arranged in a pattern for visualization using conventional visualization techniques during use.

The transection device 200 is structured for sterilization using conventional techniques such as ethylene oxide, electron beam, gamma, and autoclaving as well as chemical sterilization and aseptic packaging techniques.

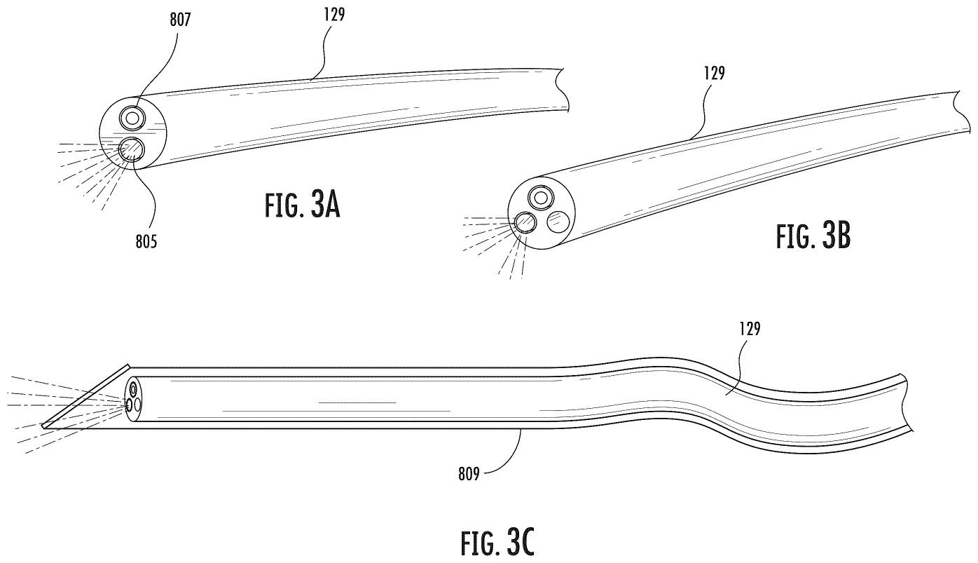

With reference to FIGS. 3A-3C, the transection device 200, in some examples, further comprises visualization means. As shown, a multi-lumen 129 is illustrated without a transection device 200 for clarity. Such a multi-lumen 129 comprises a fiber optic channel and lens 807 adjacent a fiber optic channel 805 to provide light and to provide an analog or digital image in a multi-lumen 129, which may also have a sheath 809. In one example, the optical channel in the multi-lumen accommodates a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen to accommodate a lens coupled to a fiber optic cable, optionally with a light source, e.g., an LED. In some examples, the fiber optic channel 805 may operate as the visualization means (e.g., imaging portion of the operation) as well as the light source.

As described above with reference to FIGS. 1A, 1B, and 1C, layers of a heart wall of a heart 50, with pericardium 60, from inside-out, being the endocardium 51, the myocardium 52, epicardial adipose tissue 57, the visceral layer of the serous pericardium 53, the pericardial cavity 54, the parietal layer of the serous pericardium 55, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity 54 and superficial to the visceral layer 53 of the pericardium 60.

In one example, to provide orientational stability of the cutting surface to that of the parietal layer, an OTW introduction is employed for any of the previously disclosed devices, for example, whether through a dedicated lumen in multi-lumen cross-section or ‘Rapid Exchange’ style catheter, or off-center attached cannula, or deflect-resistant catheter, as the delivering catheter randomly distributed or arranged in a pattern for visualization using conventional visualization techniques during use.

Current ECHO/fluoroscopy may not provide the required visualization for certain access applications of the presently disclosed transection devices, for example, gaining guidewire access pericardial cavity consistently and repeatedly may be desired. Thus, in one example, the multi-lumen device 129 coupled to the presently disclosed transection devices comprises direct visualization, as shown in FIGS. 3A-3C, allowing the user to watch real-time the advancement of the transection device 200 through various tissue layers until the desired location is reached. Changes in tissue layers that may not be visible under ECHO/fluoroscopy may be easily distinguishable under direct visualization such as tissue (vessel access), myocardium/pericardium (pericardial cavity access), myocardium/pericardium (outside pericardium), among other anatomical features.

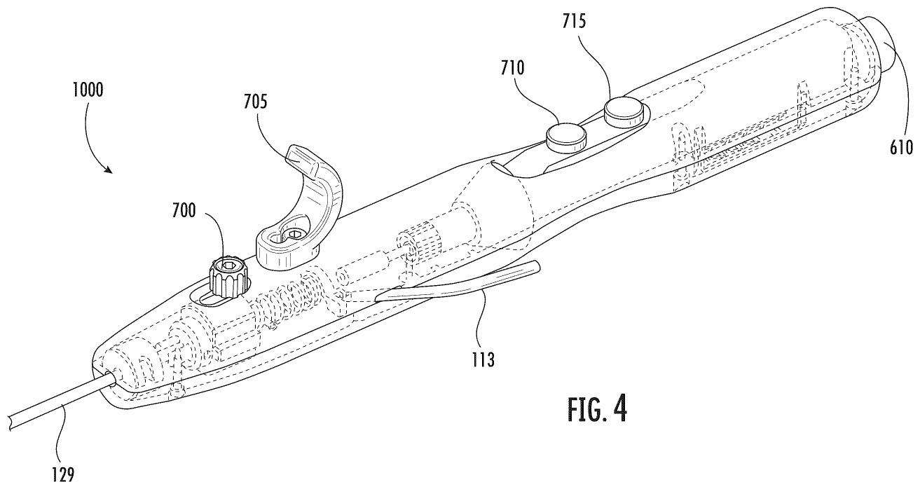

With reference to FIG. 4, the transection device 200 may be manipulated and/or controlled from outside the subject using a controller 1000, which may be a handle. The controller 1000 may have multiple actuating knobs 700, 705, 610 and actuating buttons 710, 715 for controlling the multi-lumen 129 and the various components of the transection device 200. Knob 700 may be structured to rotate the flexible catheter 129 in response to orientation information derived from fluoroscopy or other visualization means. In some examples, knob 700 may operate to cause extension/retraction of, for example, the stabilizing members 120. Knob 705 may be engaged to activate one or more components on the medical device (e.g., actuating the stabilizing member(s)). Similarly, the actuating buttons 710, 715 may be engaged to activate various components of the multi-lumen (e.g., the device may use RF electrode cutting as well and the actuating buttons 710, 715 may be used to supply current or RF). Various other controllers are envisioned that allow for the aforementioned transection devices to be deployed and manipulated. A method of manipulating transection device 200 would comprise providing pericardial device 200 engaged with controller 1000 and controlling, for example, manipulating any one or combination of multiple actuating knobs 700, 705, 610 and actuating buttons 710, 715 to provide at least one of rotation of the elongated body 129 (or steerable catheter with steerable segments), extension and retraction of the incision member 103, incision assembly 101, stabilizing members 120, and providing energy (e.g., radio frequency (RF)) to the incision member with controller 1000. The aforementioned method is applicable to any previously disclosed transection devices.

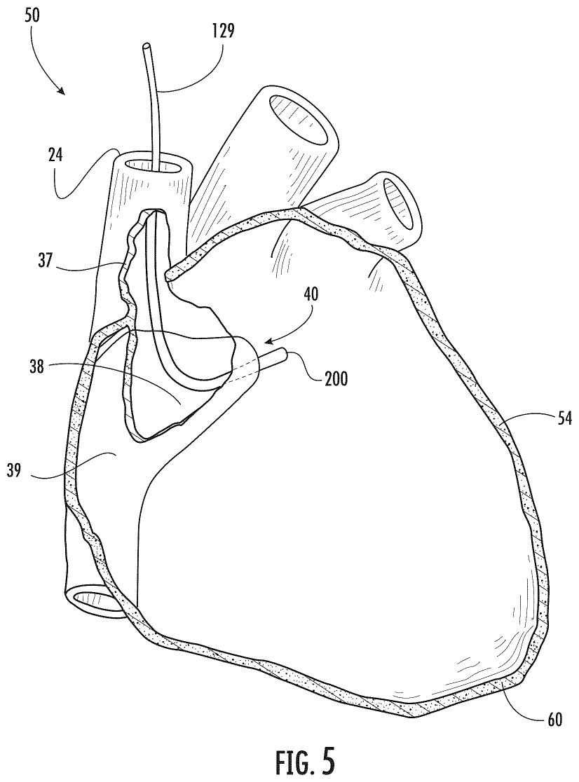

FIGS. 5-7 illustrate various intravascular approaches for delivering the transection devices of the present disclosure to the pericardial cavity 54. Thus, FIG. 5 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The presently disclosed transection device 200 may be presented to the pericardial cavity 54. In one example via the right atrial appendage 38 (RAA), a suitable site for entry into the pericardial cavity 54, is used. Right atrial appendage 38 lies tangential to and between pericardium 60 and the epicardium/epicardial adipose tissue 57. In one example, any of the presently disclosed transection devices 200 is guided into right atrial appendage 38 via right atrium 39 to be positioned substantially in parallel with the wall of pericardium 60 such that when the wall of right atrial appendage 38 is pierced by the transection device 200 it is done substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, the right atrial appendage 38 may be accessed via conventional vena cava routes. FIG. 5 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

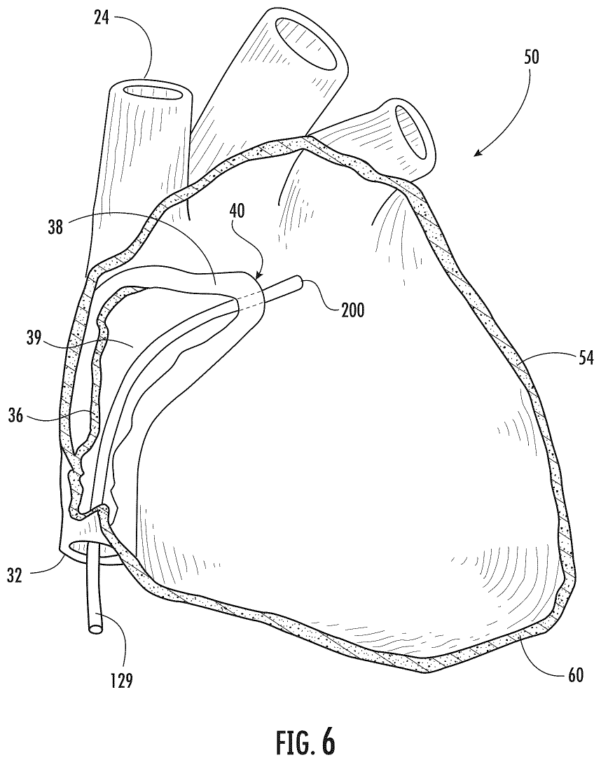

FIG. 6 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, the method of the present disclosure includes the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged.

The wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

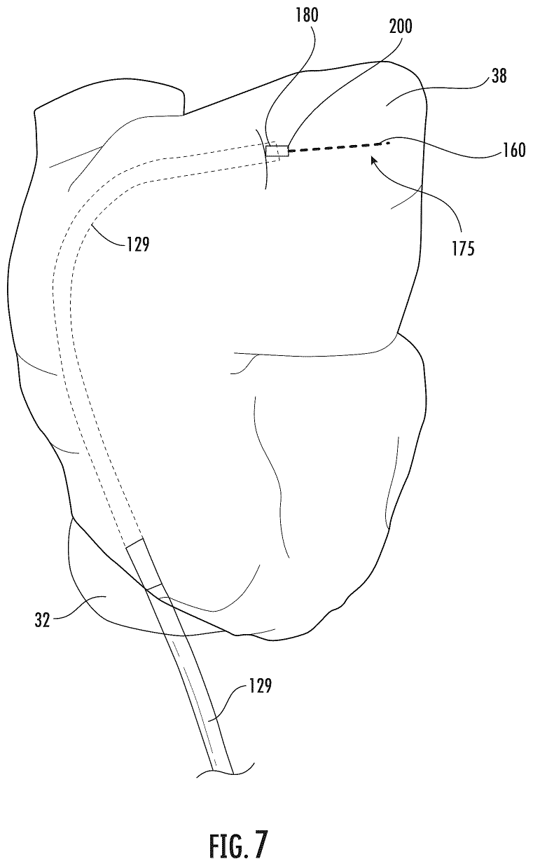

As shown in FIG. 7, a steerable catheter 129 may be employed, extending through the IVC, through the RA, and into the RAA and then into the pericardial cavity 54, the steerable catheter having a plurality of steerable segments. In some examples, the steerable catheter guiding the transection device 200 may have a radius of curvature of between about 1 inch and about 5 inches, with an arc length of between about 90° and about 180°. As shown in FIG. 7, transection device 200 begins a cut path 175 at a start point 160 and ends at endpoint 180. At least a portion of the parietal layer of the serous pericardium 55, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device further comprises at least one nerve detection device. In one example, the at least one nerve detection device is located on the flexible catheter 129. In one example, the at least one nerve detection device is located adjacent the incision assembly 101. In one example, the at least one nerve detection device is located on the introducer/dilator 115. In one example, the at least one nerve detection device is located on the incision member 103.

A kit, comprising any one of the presently disclosed medical devices, a sheath 130, a guidewire 113, and an introducer/dilator 115 is provided.

Any one of the presently disclosed devices can further comprises at least one nerve stimulation device. In one example, the at least one nerve stimulation device is located on the catheter 129. In one example, the at least one nerve stimulation device is located adjacent the incision device. In one example, the at least one nerve stimulation device is located on dilator/introducer 115. In one example, the at least one nerve stimulation device is located on the cutting surface.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures but may also encompass combinations of elements of the various illustrated examples and aspects thereof.

We claim:

1. A pericardial tissue transection device comprising:
an elongated body with a proximal end and a distal end, and extending along a longitudinal length; and
an incision assembly coupled to the distal end of the elongated body, the incision assembly comprising an incision member aligned with the longitudinal length of the elongated body and comprising respective first and second ends coupled to the incision assembly, wherein at least a portion of the incision member is structured to reversibly extend laterally from the elongated body.

2. The pericardial tissue transection device of claim 1, further comprising an actuator operably coupled to the incision assembly, the actuator traversing along the longitudinal length of the elongated body from a first configuration disengaged with the incision member to a second configuration engaged with the incision member such that in the second configuration the incision member is laterally extended from the elongated body.

3. The pericardial tissue transection device of claim 2, wherein the actuator is a rigid rod or wire.

4. The pericardial tissue transection device of claim 1, wherein the incision member is planar and rigidly flexible.

5. The pericardial tissue transection device of claim 1, further comprising an actuator operably coupled to the incision assembly and structured to operably laterally retract and extend the incision assembly from the elongated body by respectively removing and applying a force to the incision assembly.

6. The pericardial tissue transection device of claim 1, wherein at least a portion of the incision member is a sharpened edge and/or wherein at least a portion of the incision member is energizable with electrical current or radio frequency energy.

7. The pericardial tissue transection device of claim 1, wherein the elongated body comprises an opening, the incision member structured to reversibly extend laterally from the opening.

8. The pericardial tissue transection device of claim 1, further comprising one or more stabilizing members configured to reversibly extend laterally from the elongated body.

9. The pericardial tissue transection device of claim 1, further comprising:
an opening positioned on the top of incision assembly, the incision member structured to reversibly extend laterally from the opening; and
two or more stabilizing members positioned radially about the incision assembly and aligned on a bottom of the incision member.

10. The pericardial tissue transection device of claim 1, further comprising a retractable sheath covering the incision assembly.

11. The pericardial tissue transection device of claim 1, further comprising an introducer or a dilator adjacent the distal end of the incision assembly.

12. The pericardial tissue transection device of claim 10, wherein the introducer or the dilator is structured for receiving a guidewire. 13 The pericardial tissue transection device of claim 1, wherein the pericardial transection device is sterilized.

14. The pericardial tissue transection device of claim 1, further comprising a controller engaged to the incision assembly, wherein the controller is structured to provide at least one of: rotation of the elongated body, extension and retraction of the incision device, and/or to energize the incision device.

15. A pericardial tissue transection device comprising:
an elongated body with a proximal end and a distal end, and extending along a longitudinal length;
an incision assembly coupled to the distal end of the elongated body, the incision assembly comprising an incision member aligned with the longitudinal length of the elongated body and comprising respective first and second ends coupled to the incision assembly, wherein at least a portion of the incision member is structured to reversibly extend laterally from the elongated body;
stabilizing members adjacent to the incision assembly structured to reversibly extend laterally from the elongated body; and
an actuator operably coupled to the incision assembly and structured to operably laterally retract and extend the incision assembly from the elongated body by respectively removing and applying a force to the incision assembly, wherein when said actuator applies a force to the incision assembly, the incision assembly flexes laterally and extends from the elongated body.

16. The pericardial tissue transection device of claim 15, wherein at least a portion of the incision member is a sharpened edge or wherein at least a portion of the incision member is energizable with electrical current or radio frequency energy.

17. A pericardial tissue transection device comprising:
an elongated body with a proximal end and a distal end, and extending along a longitudinal length;
an incision assembly coupled to the distal end of the elongated body, the incision assembly comprising an incision member aligned with the longitudinal length of the elongated body and comprising respective first and second ends coupled to the incision assembly, wherein at least a portion of the incision member is structured to reversibly extend laterally from the elongated body;
two or more stabilizing members positioned radially about the incision assembly and aligned on a bottom of the incision member and to reversibly extend laterally from the elongated body; and
an actuator operably coupled to the incision assembly and structured to operably laterally retract and extend the incision assembly from the elongated body by respectively removing and applying a force to the incision assembly, wherein when said actuator applies a force to the incision assembly, the incision assembly flexes laterally and extends from the elongated body.

18. The pericardial tissue transection device of claim 17, further comprising:
an opening positioned on the top of incision assembly, the incision member structured to reversibly extend laterally from the opening.

19. The pericardial tissue transection device of claim 17, wherein at least a portion of the incision member is a sharpened edge and/or wherein at least a portion of the incision member is energizable with electrical current or radio frequency energy.

20. The pericardial tissue transection device of claim 17, further comprising a retractable sheath covering the incision assembly.
